(19) United States
(12) Patent Application Publication
(10) Pub. No.: US 2023/0215551 A1
(43) Pub. Date: Jul. 6, 2023

(54) MACHINE LEARNING FOR RESOURCE ALLOCATION

(71) Applicant: MatrixCare, Inc., Bloomington, MN (US)

(72) Inventors: Vivek KUMAR, Eden Prairie, MN (US); Coleen Patrice DANIELSON, Fergus Falls, MN (US)

(21) Appl. No.: 18/061,178

(22) Filed: Dec. 2, 2022

Related U.S. Application Data
(60) Provisional application No. 63/295,351, filed on Dec. 30, 2021.

(51) Int. Cl. G16H 40/20

(52) U.S. Cl. CPC G16H 40/20

(57) ABSTRACT

Techniques for improved resource allocation via machine learning are provided. A set of resident characteristics for a first residential facility is received. A future staffing allocation is generated by processing the set of resident characteristics using one or more trained machine learning models, and modification of future staffing of the first residential facility is initiated based on the future staffing allocation.

### CROSS-REFERENCE TO RELATED APPLICATIONS

This application claims benefit of co-pending U.S. provisional patent application Ser. No. 63/295,351 filed Dec. 30, 2021. The aforementioned related patent application is herein incorporated by reference in its entirety.

### INTRODUCTION

Embodiments of the present disclosure relate to machine learning. More specifically, embodiments of the present disclosure relate to using machine learning to improve resource allocation.

In a wide variety of medical (and non-medical) settings, allocation of various resources, including human resources, is an incredibly complex problem. For example, the number of staff needed for a given facility and time can vary dramatically based on user needs, relevant regulations, and the like. As these variables naturally change over time (and, at times, change rapidly), the optimal staffing is equally dynamic. Moreover, in addition to determining the proper amount of staff, the proper mix of staff (e.g., with various skills and specialties) adds exponentially increased complexity.

Improved systems and techniques to evaluate resource allocation and generate improved or optimized allocations are needed.

### SUMMARY

According to one embodiment presented in this disclosure, a method is provided. The method includes: determining, for one or more prior times, a historical set of resident characteristics for a first residential facility; determining, for the one or more prior times, historical staffing allocations for the first residential facility; and training one or more machine learning models, based on the historical set of resident characteristics and the historical staffing allocations, to generate future staffing allocations.

According to one embodiment presented in this disclosure, a method is provided. The method includes: receiving a set of resident characteristics for a first residential facility; generating a future staffing allocation by processing the set of resident characteristics using one or more trained machine learning models; and initiating modification of future staffing of the first residential facility based on the future staffing allocation.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for training one or more machine learning models to generate optimized resource allocations.

FIG. 2 depicts an example environment for using one or more machine learning models to generate optimized resource allocations.

FIG. 3 depicts an example environment for using one or more machine learning models to implement optimized resource allocations.

FIG. 4 depicts an example workflow for determining resident characteristics to enable generation of optimized resource allocations.

FIG. 5 depicts an example workflow for determining historical staffing to enable generation of optimized resource allocations.

FIG. 6 is a flow diagram depicting an example method for training machine learning models to generate optimized resource allocations.

FIG. 7 is a flow diagram depicting an example method for determining historical staffing to enable generation of optimized resource allocations.

FIG. 8 is a flow diagram depicting an example method for determining resident characteristics to generate optimized resource allocations.

FIG. 9 is a flow diagram depicting an example method for evaluating historical data to improve training machine learning models to generate optimized resource allocations.

FIG. 10 is a flow diagram depicting an example method for generating optimized resource allocations using machine learning models.

FIG. 11 is a flow diagram depicting an example method for implementing optimized resource allocations using machine learning models.

FIG. 12 is a flow diagram depicting an example method for generating optimized resource mixes using machine learning models.

FIG. 13 is a flow diagram depicting an example method for training machine learning models to generate future staffing.

FIG. 14 is a flow diagram depicting an example method for using trained machine learning models to modify future staffing.

FIG. 15 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning models for resource allocation.

Resource allocation is a tremendously impactful problem for a wide variety of industries and facilities. If too many resources are allocated, waste occurs as some portion of the allocated resources are unused or underutilized. If too few are allocated, the functioning of the system can significantly degrade as insufficient resources are provided to handle the workload. In some embodiments discussed herein, staffing resources (e.g., human employees) are used to provide concrete examples of resource allocation for conceptual clarity. However, embodiments of the present disclosure are readily applicable to a wide variety of applications.

In some embodiments, machine learning models are trained and used to generate optimized staffing allocations based on facility-specific data (e.g., characteristics of the residents in a residential facility, such as a nursing home). For example, based on data such as the average age of the residents, daily assistance needed, medications needed by the residents, and the like, machine learning can be used to generate an optimized allocation of staffing resources, which may include a number of staff, the skill mix of the staff, and the like.

In embodiments, by training and using unique machine learning models, the system is able to dynamically adjust resource allocation based on changing conditions. This can reduce resource waste, improve facility efficiency, improve the individual care and aid offered to specific residents and users, and the like.

### Example Environment for Training Machine Learning Models to Generate Optimized Resource Allocations

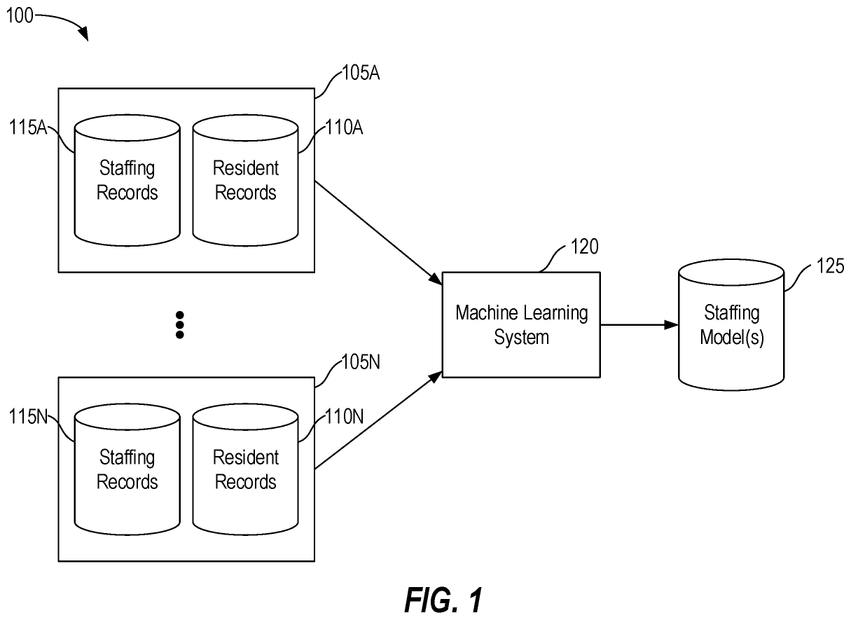

FIG. 1 depicts an example environment 100 for training one or more machine learning models to generate optimized resource allocations.

In the illustrated environment 100, a machine learning system 120 receives data corresponding to a number of facilities 105A-N. Though two facilities 105 are depicted in the illustrated example, in embodiments, there may be any number of facilities in a given deployment. Generally, each facility 105 corresponds to a particular physical location where resources are required. For example, each facility 105 may correspond to a residential care facility where users or patients reside for long-term care. The needed resources may include, for example, staffing resources, medical resources (e.g., medications and medical equipment), dietary resources (e.g., food), and the like. In the illustrated environment 100, staffing resources are used as an example resource that is allocated to the facilities 105.

In embodiments, the machine learning system 120 generally corresponds to one or more components that train and/or use machine learning models to generate optimized resource allocations. The machine learning system 120 may generally be implemented using hardware (e.g., as one or more physical systems), software (e.g., in a cloud deployment), or a combination of hardware and software. Though the illustrated environment 100 includes a single machine learning system 120 that receives data for each facility 105, in some embodiments, there may be a separate machine learning system 120 associated with each facility 105. That is, a separate machine learning system 120 may be used to train models specific to each facility 105, and these specific models may be used to optimize resource allocation at the corresponding facility. Similarly, in some aspects, the machine learning system 120 may process data from all facilities 105, but generate specific models targeted to each specific facility 105.

In embodiments, the machine learning system 120 receives various data from each facility 105 to train machine learning models. In the illustrated example, the machine learning system 120 can receive or determine, for each facility 105, a set of staffing record(s) 115 and a set of resident record(s) 110. For other resource allocations (e.g., for medical supplies), other data may be used in various embodiments.

In one embodiment, the staffing records 115 can generally include data relevant to the staffing of the corresponding facility 105 at one or more prior points or windows in time (e.g., during one or more prior shifts that have ended). For example, in some embodiments, the staffing records 115 can indicate the number of staff that worked a given shift, the skill mix of the present staff (e.g., the number of base caregivers, the number of medication specialists, and the like), or both.

In some embodiments, the actual staff allocation may not be readily available in the data. For example, a given facility 105 may not track the specific staff allocation for past shifts. In at least one embodiment, the machine learning system 120 (or another intermediary component) can evaluate other data to determine the prior allocations. For example, in one such embodiment, the received data may include some indication of the charts used in the facility 105. As used herein, the facility charts generally correspond to notes, narratives, or other information indicating what work was performed and who completed the work. For example, a chart may indicate, for a given resident, what treatments or interventions were administered, when they were administered, what staff member performed them, and the like.

In one such embodiment, to determine the prior staffing, the machine learning system 120 can evaluate these charts for the facility and time (e.g., recorded charts for a given shift) to determine the number of employees (and, in some cases, their skill mix) that were present for the given shift. For example, given a set of charts indicating all actions that were performed during the shift, the machine learning system 120 can identify all unique employees that logged at least one action during the shift, and aggregate this data to determine the number of employees (of each skill type) that worked the shift. In at least one embodiment, prior to this evaluation, the data can be anonymized. For example, the actual contents of each chart (e.g., the particular treatments administered, as well as the patients to whom treatment was provided) may be obfuscated or removed from the data. This can allow the machine learning system 120 to infer the staff allocation for the shift without potentially compromising resident privacy.

In some embodiments, the staffing records 115 may additionally or alternatively include data that can be used (e.g., by the machine learning system 120, or by another component) to determine or infer whether the prior staffing allocation was appropriate. For example, in one such embodiment, the data may explicitly indicate, for one or more shifts, whether staffing was adequate or appropriate (e.g., recorded by one or more of the staff, by a manager of the shift, and the like). If staffing was inappropriate, in one such embodiment, the data can indicate what should have been corrected (e.g., whether more or fewer people were needed overall, whether more or fewer people with specific skills were needed, and the like).

In some embodiments, the machine learning system 120 can infer whether staffing was appropriate based on the aforementioned charts. In one such embodiment, the machine learning system 120 can determine the number residents being served by a single employee during the shift. For example, the machine learning system 120 may evaluate the charts to determine, for each staff member on the shift, the number of different residents served by the staff member, and/or the number of different interventions or assistance events that were performed by the staff member.

By comparing this resident-to-staff ratio (or assistance events-to-staff ratio) to defined thresholds (e.g., policies indicating that a given staff member should be expected to serve no more than five residents per shift), the machine learning system 120 can determine whether the staffing was appropriate. For example, if the average ratio is below the threshold, the machine learning system 120 can determine that excess staff was present, and that fewer would have been able to perform the same work. If the ratio exceeds the threshold, the machine learning system 120 may determine that additional staff were needed or would have been advantageous.

In some embodiments, the machine learning system 120 can additionally or alternatively infer whether the prior staffing was appropriate based on data such as overtime records for the facility. For example, the machine learning system 120 may determine the number or percentage of staff members that regularly work overtime (e.g., at least a defined percentage of days or weeks), the number or percentage of overtime hours worked in the facility (as compared to the number of non-overtime hours), and the like. If these overtime hours exceed a defined threshold (e.g., a maximum target), the machine learning system 120 can infer that the facility is understaffed and that additional staff would be advantageous.

Regardless of the particular technique(s) used to determine the historical staffing, in the illustrated environment 100, the machine learning system 120 can use these staffing records 115 (potentially adjusted based on whether the staffing was adequate) to train a set of staffing models 125. For example, the machine learning system 120 may use the (potentially modified) staffing records as target output for the staffing models 125, as discussed in more detail below.

In the illustrated environment 100, the machine learning system 120 also receives resident record(s) 110 from each facility 105. The resident records can generally indicate various characteristics of the residents of the facility 105, such as activities and daily living (ADL) needs, behavioral issues in the facility 105, resident(s) that are high risk (e.g., for infection or fall), isolation requirements (e.g., the number of residents in isolation due to infection or illness), vitals such as the weight and height of one or more residents or the average age of the residents, medication requirements (e.g., the number of medication orders per shift, the types of medication orders, and the like), the number of new residents in the facility 105, reported pain concerns among the residents, treatment requirements (e.g., needed interventions, physical therapy, and the like) for the residents, wound care needed (e.g., the number of residents with open wounds needing care), acuity scores for the residents (e.g., the number of residents having significant care needs), and the like.

Generally, the resident records 110 may be used to determine the appropriate staffing for the facility 105. For example, the number of ADL events, treatment events, wound care events, medication events, high risk residents, high acuity residents, new residents, and the like can all be positively correlated with the number of staff needed. Similarly, the mix of these events (e.g., the number of ADL events and medication events) can inform the appropriate skill mix of the staff (e.g., the number of base caregivers and medication aides). However, the massive number and diversity of these records renders them entirely opaque to human users.

Therefore, in the illustrated example, the machine learning system 120 can train a set of one or more machine learning models (e.g., staffing models 125) based on the staffing records 115 and resident records 110 for one or more facilities 105. For example, in one embodiment, the resident characteristics for a given facility 105 during a given shift can be provided as input to a model undergoing training. Based on this input, the model can output a staffing allocation (which may include a number and/or mix of staff for the shift). During training, particularly in early phases, this prediction may be relatively inaccurate or unreliable (e.g., because the model may be initiated with random or pseudo-random values).

During training, the machine learning system 120 can compare the generated allocation to the actual allocation (e.g., determined based on the staffing records 115) for the facility and shift. For example, the machine learning system 120 can compute a loss based on the difference, and use this loss to refine the weights, biases, and other parameters of the staffing model(s) 125. In embodiments, this training process can be performed iteratively for each shift in each facility (e.g., using stochastic gradient descent), or collectively for multiple shifts and/or facilities simultaneously (e.g., using batch gradient descent).

In some embodiments, as part of this training process, the machine learning system 120 can determine whether the prior staffing was appropriate, prior to using it to refine the staffing model(s) 125. If such evaluation is not performed, the staffing models 125 may learn based on non-optimized inputs (e.g., inadequate or excessive staffing), which can result in sub-optimal output predictions. For example, the machine learning system 120 may mistakenly suggest more or fewer staff than needed. In one embodiments, therefore the machine learning system 120 may discard data from any shifts where it is determined that the staffing was inadequate or excessive. This allows the machine learning system 120 to train using only good data, thereby improving the model performance.

In at least one embodiment, rather than discarding the data from these poorly-staffed shifts, the machine learning system 120 can modify the prior staffing records prior to training the model. For example, if the machine learning system 120 determines or infers that too few medication specialists were present, the machine learning system 120 can use a relatively larger number of medication specialists as target output for the training cycle. Similarly, if the machine learning system 120 determines or infers that too many base caregivers were present, the machine learning system 120 can use a relatively lower value as target output. This can allow the machine learning system 120 to use more (or all) of the available training data, which can significantly improve model accuracy. Additionally, by modifying the data first, the machine learning system 120 ensures that the resulting allocations are more reliable and accurate.

In some embodiments, the staffing models 125 may be retrained or refined periodically or upon specified events. For example, the machine learning system 120 may retrain or refine the models weekly based on updated data from the past week. In some embodiments, the machine learning system 120 can refine the staffing models 125 whenever it is determined that a generated allocation was inapt. For example, suppose the machine learning system 120 generates a proposed allocation for a given shift. If it is determined that this allocation is excessive or inadequate, the machine learning system 120 can refine the models based on this determination. This determination may be made in a variety of ways, including by a user (e.g., a manger) prior to, during, or after the shift, or by the machine learning system 120 (e.g., by evaluating the charting ratios, overtime hours, and the like after the shift).

In some embodiments, the machine learning system 120 uses the trained staffing models 125 to generate resource allocations. That is, the machine learning system 120 may both train the models, as well as use them for inferencing. In other embodiments, the models may be trained by the machine learning system 120, and deployed to one or more other components or systems for inferencing.

### Example Environment for Using Machine Learning to Generate Optimized Resource Allocations

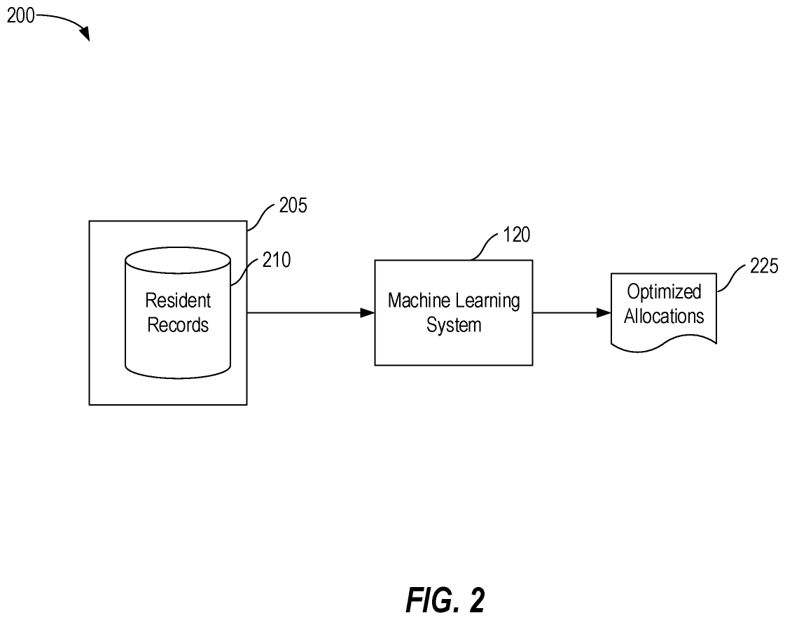

FIG. 2 depicts an example environment 200 for using one or more machine learning models to generate optimized resource allocations.

In the illustrated environment 200, a machine learning system 120 receives data corresponding to a number of facilities 205. Though one facility 205 is depicted in the illustrated example, in embodiments, there may be any number of facilities in a given deployment. Generally, as discussed above, the facility 205 corresponds to a particular physical location where resources are required. For example, the facility 205 may correspond to a residential care facility where users or patients reside for long-term care. Further, as discussed above, the needed resources may include, for example, staffing resources, medical resources (e.g., medications and medical equipment), dietary resources (e.g., food), and the like. In the illustrated environment 200, as with the environment 100, staffing resources are used as an example for conceptual clarity.

In embodiments, as discussed above, the machine learning system 120 generally corresponds to one or more components that train and/or use machine learning models to generate optimized resource allocations. The machine learning system 120 may generally be implemented using hardware (e.g., as one or more physical systems), software (e.g., in a cloud deployment), or a combination of hardware and software.

Though the illustrated example includes the machine learning system 120, in some embodiments, as discussed above, a separate system may be used to generate allocations using the trained models (e.g., trained by the machine learning system 120 of FIG. 1). In some embodiments, the machine learning system 120 can use a set of one or more machine learning models (e.g., staffing models 125 of FIG. 1) to generate optimized allocations 225 for the facility 205.

In some embodiments, a unique set of model(s) are used for each facility 205. That is, the machine learning system 120 may train or refine a separate model for each facility 205, allowing each facility 205 to be processed using a corresponding and uniquely-trained model. In other embodiments, the machine learning system 120 can share the model(s) across all facilities 205, generating optimized allocations 225 for each using the global model. In at least one embodiment, a global model can be trained for multiple facilities, and the global model can be refined using data for each individual facility 205 to generate a specialized model for each facility.

In embodiments, the facility 205 may or may not have been involved in the training of the models. That is, prior data from the facility 205 may or may not have been collected and used to refine the models (e.g., as discussed above with reference to FIG. 1). For example, the model(s) may be trained based in part on data from the facility 205, and the models can be used to generate optimized allocations for the facility. Alternatively, the models may be trained based only on data from one or more other facilities, and these models can then be deployed to generate optimized allocations for the facility 205.

As illustrated, in the environment 200, the machine learning system 120 receives data from the facility 205, to be evaluated (e.g., using trained machine learning models) to generate the optimized allocations 225. In the illustrated example, the machine learning system 120 can receive or determine a set of resident record(s) 210. For other resource allocations (e.g., for medical supplies), other data may be used in various embodiments.

In some embodiments, as discussed above, the resident records 210 can generally indicate various characteristics of the residents of the facility 205. In some embodiments, the resident records 210 indicate the current characteristics (e.g., for the current shift or an imminent shift), and/or the expected characteristics for one or more shifts in the future. For example, the resident records 210 may indicate the expected medication orders needed for a shift that is about to begin, for a shift that begins in a few days, and the like.

As discussed above, the resident records 210 may generally include characteristics such as activities and daily living (ADL) needs, behavioral issues in the facility 205, resident(s) that are high risk (e.g., for infection or fall), isolation requirements (e.g., the number of residents in isolation due to infection or illness), the average age of the residents, medication requirements (e.g., the number of medication orders per shift, the types of medication orders, and the like), the number of new residents in the facility 205, reported pain concerns among the residents, treatment requirements (e.g., needed interventions, physical therapy, and the like) for the residents, wound care needed (e.g., the number of residents with open wounds needing care), acuity scores for the residents (e.g., the number of residents having significant carte needs), and the like.

As discussed above, the resident records 210 may generally be selected and/or evaluated because they may be used to determine the appropriate staffing for the facility 205. For example, the number of ADL events, treatment events, wound care events, medication events, high risk residents, high acuity residents, new residents, and the like can all be positively correlated with the number of staff needed. Similarly, the mix of these events (e.g., the number of ADL events and medication events) can inform the appropriate skill mix of the staff (e.g., the number of base caregivers and medication aides).

However, as discussed above, the massive number and diversity of these records renders them entirely opaque to human users. Therefore, in the illustrated example, the machine learning system 120 uses one or more trained machine learning models to generate a set of optimized allocations. For example, in the case of staffing allocation, the optimized allocations 225 may indicate the optimized allocations 225 may indicate the total number of staff that should be allocated, the skill mix of the staff (e.g., the number of specialists for each possible specialty), and the like.

As used herein, these proposed, recommended, or suggested allocations may be referred to as “optimized” to indicate that they were generated by the trained machine learning model(s), and therefore may be less likely to result in inadequate or excessive coverage. In this way, the optimized allocations 225 can improve the care provided to the residents in the facility, reduce waste of resources (including staffing time, as well as other resources such as medical equipment), and the like. For example, based on the specific needs of one or more residents at a specific facility, the machine learning system 120 can generate a specific allocation of resources that ensure the resident needs are met accurately and completely, and without waste.

In some embodiments, the machine learning system 120 can use a single machine learning model to generate the optimized allocations 225. For example, a single model may generate resource allocations for a number of resources (e.g., the number of basic caregivers, the number of therapists, the number of medication specialists, and the like). In some embodiments, a set of models can be used (e.g., one for each resource type). For example, a first model may indicate the number of dietary aides that should be staffed, while a second model may indicate the number of medication aides that should be staffed.

In an embodiment, the optimized allocations 225 correspond to suggested resource allocation for a given period of time (e.g., for the shift that corresponds to the resident records 210). In this way, the machine learning system 120 can generate new optimized allocations 225 for each relevant window of time, enabling the system to dynamically adjust these allocations to account for changing circumstances and ensure proper load balancing (e.g., distributing staff or other resources appropriately).

### Example Environment for Using Machine Learning to Implement Optimized Resource Allocations

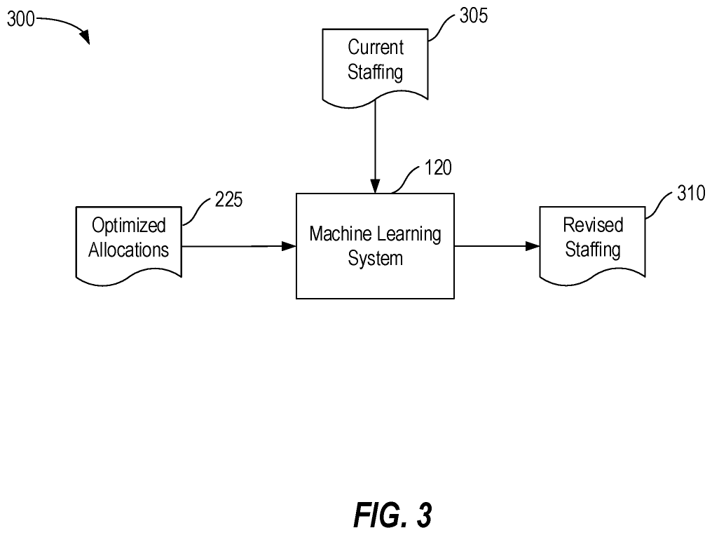

FIG. 3 depicts an example environment 300 for using one or more machine learning models to implement optimized resource allocations.

In the illustrated environment 300, a machine learning system 120 receives a set of optimized allocations 225 for one or more periods of time at one or more given facilities. For example, as discussed above with reference to FIG. 2, the optimized allocations 225 may be generated based on resident data to ensure adequate and appropriate allocation of scarce resources. Though staffing allocations are used for conceptual clarity in some embodiments, as discussed above, the needed resources may include a variety of resources, including staffing resources, medical resources (e.g., medications and medical equipment), dietary resources (e.g., food), and the like.

In the illustrated embodiment, the machine learning system 120 uses the optimized allocations 225 to evaluate and modify the actual staffing of the facility or facilities. In some embodiments, this process is performed by a separate component or system, discrete from the system that trains the model(s) and/or uses the model(s) to generate the optimized allocations 225. As discussed above, the machine learning system 120 may generally be implemented using hardware (e.g., as one or more physical systems), software (e.g., in a cloud deployment), or a combination of hardware and software.

As discussed above, the optimized allocations 225 may generally include suggested or proposed resource allocations for a given window of time and/or facility. For example, for a given shift at a residential care facility, the optimized allocations 225 may indicate the optimal number of basic caregivers, medication aides, dietary aides, and the like. In an embodiment, as discussed above, the optimized allocations 225 are generated based on resident-specific characteristics of the facility during the given window, such as the expected number of ADL events, the number of medication orders that will be administered, and the like.

In the illustrated example, the machine learning system 120 can also receive and evaluate the current staffing 305 for the facility, at the relevant time. For example, if the optimized allocations 225 indicate the suggested staffing for a shift that begins the next morning (e.g., at 9:00 am) at Facility A, the machine learning system 120 can retrieve the current staffing 305 (e.g., the scheduled allocation of staff) for Facility A during that shift.

As illustrated, based on the current staffing 305 and the optimized allocations 225, the machine learning system 120 generates revised staffing 310 for the facility. In one such embodiment, if the current staffing 305 is below the optimized allocations 225, the revised staffing 310 can indicate an increase in the relevant areas. For example, if the current staffing 305 allocates five basic caregivers, but the optimized allocations 225 suggest seven, the machine learning system 120 can generate a revised staffing 310 indicating that two additional caregivers should be staffed for the shift, if possible.

In some embodiments, the revised staffing 310 has the same granularity as the optimized allocations 225 and/or current staffing 305. For example, if the optimized allocations 225 indicate both a number of base caregivers and a number of medication aides, the revised staffing 310 can indicate whether any changes are needed for these specialties. In one embodiment, if the current staffing 305 simply indicates the total number of employees, the revised staffing 310 may indicate an overall increase or decrease (if relevant). In an embodiment, the revised staffing 310 can generally include both increased resource allocation for some resources, as well as decreased allocations for others. For example, the revised staffing 310 may indicate to allocate an additional therapist and remove one medication specialist from the staffing for the given shift.

In this way, the machine learning system 120 can dynamically modify the scheduling. In some embodiments, the machine learning system 120 can implement the revised staffing 310 entirely or partially automatically. In some embodiments, a human may be required to review and/or approve some or all of these changes. For example, in one such embodiment, the machine learning system 120 may be authorized to remove staff from the schedule automatically, but adding staff may require approval by a human. In other embodiments, removing staff from the shift may require approval, while adding staff can be performed automatically.

In some embodiments, in addition to (or instead of) identifying the staff changes that are warranted, the revised staffing 310 may also indicate or suggest particular employees. For example, if additional staff are needed, the machine learning system 120 may suggest specific employees to fill those roles (e.g., based on seniority, availability, their current or preferred number of hours worked per week, and the like).

In at least one embodiment, the machine learning system 120 can additionally or alternatively contact one or more external agencies (e.g., staffing agencies) to request resources indicated by the revised staffing 310. For example, if the revised staffing 310 indicates that three more nurses are needed, but there are no additional nurses available to be staffed, the machine learning system 120 may contact a nursing agency to request that three nurses (with the appropriate skillset) be sent to cover the indicated shift at the indicated facility.

### Example Workflow for Determining Resident Characteristics to Enable Generation of Optimized Resource Allocations

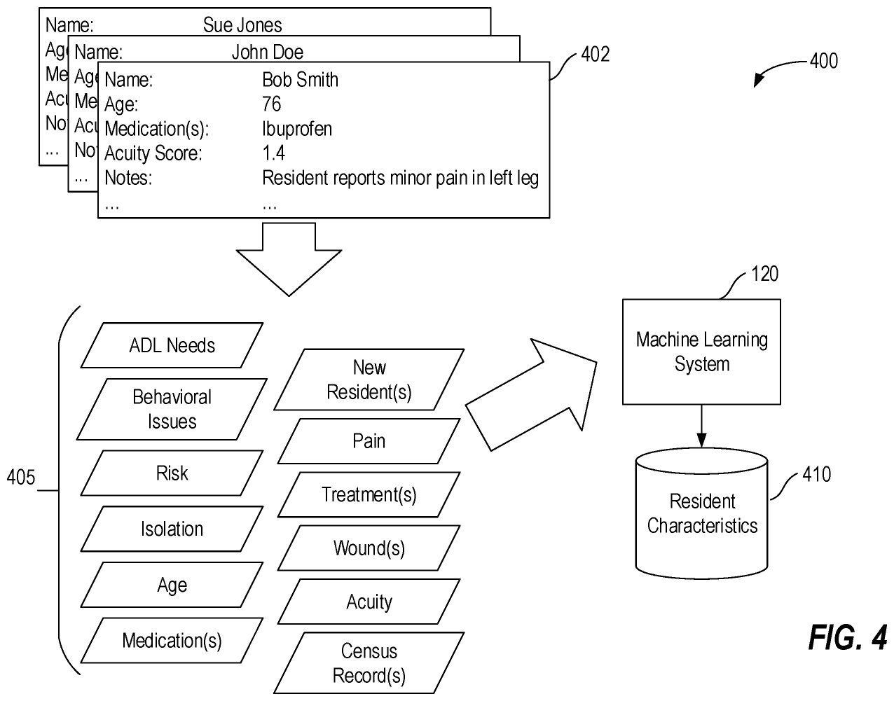

FIG. 4 depicts an example workflow 400 for determining resident characteristics to enable generation of optimized resource allocations. In some embodiments, the workflow 400 is performed by a machine learning system, such as machine learning system 120 of FIG. 1.

In the illustrated workflow 400, a diverse set of inputs 405 are received, evaluated, and/or aggregated by the machine learning system 120 in order to generate an overall set of resident characteristics 410. In some embodiments, the inputs 405 are received from one or more facilities. For example, the inputs 405 may correspond to the resident records 110 of FIG. 1.

In the depicted embodiment, the inputs 405 are extracted from one or more resident records 402. As discussed above, each resident record generally includes information relating to the residents in a facility, such as their name, age, and the like. In the illustrated example, the resident records 402 include a record for a “Sue Jones” resident, a record for a “John Doe” resident, and a record for a “Bob Smith” resident. The “Bob Smith” resident record 402 indicates the resident name, age, medication(s) the resident receives, and acuity score (e.g., generated using one or more machine learning models, as discussed above). In embodiments, the resident records 402 may include significantly more information where relevant, such as whether the resident is in isolation, their risk score(s), and the like. As indicated by the ellipses, the resident record 402 can generally include any number of additional entries.

Additionally, in the illustrated example, the resident record 402 includes a “Notes” entry, where caregivers can enter various notes relating to the care needs of the resident. As illustrated, the notes indicate that Bob Smith reports minor pain in their left leg. In at least one embodiment, as discussed above, the machine learning system 120 can use natural language processing to evaluate the notes in order to generate the inputs 405. In this example, the machine learning system 120 may determine that the “pain” input 405 should indicate the minor pain that Bob reported.

In one embodiment, the resident characteristics 410 correspond to the particular characteristics during a given shift and at a given facility. That is, the machine learning system 120 may parse the inputs 405 to extract the relevant data for a specific facility and window of time and generate resident characteristics 410 for that window and facility. The machine learning system 120 can similarly parse the inputs 405 to generate characteristics for other periods or facilities. Though the illustrated example depicts the machine learning system 120 receiving relatively raw inputs 405 to determine the resident characteristics 410, in some aspects, the inputs 405 are parsed by other systems (e.g., locally at each facility) to provide the resident characteristics 410 to the machine learning system 120.

In some embodiments, the resident characteristics 410 are used as input for the machine learning models discussed above. For example, during training, the resident characteristics 410 can be used as input to the model to generate some output allocation, while corresponding staffing allocations are used as target output against which the generated allocation is compared. During inferencing, the resident characteristics 410 can be used as input to the model to generate a corresponding output allocation, which can be used to drive staffing decisions and other resource allocations, as discussed above.

In the illustrated example, the inputs 405 include a wide variety of data. The depicted inputs 405 are included as examples for conceptual clarity, and the particular inputs used in various embodiments may differ depending on the particular application. That is, depending on the specific deployment and implementation of the techniques described herein, the machine learning system 120 may evaluate all, a subset of, or none of the depicted inputs 405. Additionally, in some aspects, other inputs may be used depending on the particular problem space and implementation.

In some embodiments, some or all of the inputs 405 may be stored or otherwise available in one or more repositories, such as a Minimum Data Set (MDS) system. As used herein, an MDS system corresponds to a process for clinical assessment of the residents in a residential facility (e.g., a nursing home). In some aspects, MDS systems may be mandated by federal law for any Medicare or Medicaid certified facility. Generally, the MDS system involves a comprehensive and standardized assessment of each resident's functional capabilities and health needs. These assessments may generally be conducted by the facility staff (e.g., trained nursing home clinicians) at various times, including at admission and/or discharge, as well as at other intervals (e.g., quarterly, annually, or when residents experience a significant change in status). In some embodiments, the MDS can generally capture information about the comorbidities of each resident, physical, psychological and psychosocial functioning of each resident, any treatments (e.g., hospice care, oxygen therapy, chemotherapy, dialysis) or therapies (e.g., physical, occupational, speech, restorative nursing) received by each resident, and the like.

In the illustrated example, the inputs 405 include “ADL Needs,” “Behavioral Issues,” “Risk,” “Isolation,” “Age,” “Medication(s),” “New Resident(s),” “Pain,” “Treatment(s),” “Wound(s),” “Acuity,” and “Census Record(s),” each of which is discussed in more detail below, in turn.

In one embodiment, the “ADL needs” input 405 corresponds to any activities and daily living (ADL) needs of the residents of a facility. Generally, these needs correspond to events or activities that the resident needs employee (e.g., caregiver) assistance performing. For example, some residents require assistance walking, transferring to and from bed, chairs, standing or seated positions, and the like. Some other examples include toileting (e.g., assisting the resident use the bathroom and/or shower or bath), eating, and the like. In at least one embodiment, the “ADL needs” input generally indicates the magnitude of these ADL needs (e.g., for a given shift and facility). For example, the ADL needs may indicate the expected number of such events (in the aggregate or per-resident), the type(s) of these events (e.g., eating, walking, transferring), the number of events belonging to each type, and the like. Generally, a higher number or complexity of ADL needs correlates with a higher staffing need.

In some embodiments, the “behavioral issues” input 405 includes information relating to any known (e.g., common) or expected behavioral issues among the residents, including, for example, outbursts, fights, and the like. In one such embodiment, this input can indicate, for example, the number of residents having such issues, the percentage of residents having such issues, the aggregate magnitude or severity of such issues, and the like. Generally, a higher number or magnitude of such issues is correlated with a higher staffing need.

In one embodiment, the “risk” input 405 corresponds to various known or expected risks among the residents, such as fall risks, infection risks, and the like. In some embodiments, the risk input 405 is determined using other systems or models. For example, in at least one embodiment, one or more machine learning models are used to generate such risk scores for each resident by processing various resident data for the corresponding resident. In embodiments, the risk input can generally indicate the aggregate scale or magnitude of such risks. For example, this input may indicate the average risk score among the residents, the number of residents with a risk score exceeding one or more defined thresholds, and the like. Generally, a higher value for the risk input is correlated with a higher staffing need.

In an embodiment, the “isolation” input 405 includes information relating to any isolation or quarantine needs of the residents. For example, due to immune system concerns (e.g., immunosuppressed residents) or contagious illnesses such as the flu, various coronaviruses, tuberculosis, and the like, residents may be isolated and/or quarantined separately from other residents. In an embodiment, these isolated residents may generally require additional staffing. For example, for some illnesses, laws or regulations may require some minimum number of dedicated staff per isolated resident. In some embodiments, regulations or policies may prevent staff that care for isolated residents from interacting with other residents. In embodiments, the “isolation” input generally indicates the number and/or severity of this isolation. For example, this input may indicate the number of residents in isolation, the number in quarantine, and the like. Generally, a higher value for this input is correlated with a higher staffing need.

In some embodiments, the “age” input 405 includes information relating the ages of the residents. For example, in one such embodiment, this input indicates the average age of the residents in the facility. Generally, the average age is positively correlated with staffing needs, as older residents tend to require, on average, additional care, as compared to younger residents. In some embodiments the age input 405 can correspond more broadly to a “vitals” input, where the vitals include data such as weight, height, and the like. For example, this input may indicate the average weight or BMI of the residents, the number of residents with a weight or BMI above or below some threshold(s), and the like.

In one embodiment, the “medications” input 405 includes information relating to the medication(s) used by the residents. In at least one embodiment, this input indicates the number of medication events throughout a window of time (e.g., during each shift). As used herein, a medication event corresponds to a time when the staff of the facility (e.g., a medication aide) must deliver medication to a resident, and/or ensure that the resident takes the medication. In some embodiments, the medication input can additionally or alternatively indicate the type of the event. For example, the input may indicate whether each event corresponds to a generic or over-the-counter medication such as ibuprofen, whether it involves administration of a psychotropic drug or some other controlled substance (such as opioids), whether it requires additional skill or time, such as an intravenous (IV) medication, and the like. In some embodiments, the medication input generally indicates the number and/or type of these events (or the number of each type). Generally, a higher number of medication events, as well as a higher complexity of such events, is positively correlated with higher staffing needs.

In an embodiment, the “new residents” input 405 includes information relating to any residents that are newly-arrived at their facility (e.g., residents that arrived within the last 24 hours, 48 hours or some other defined time). Frequently, such new residents have difficulty settling in to the facility, and may require additional aid. In some embodiments, the new resident input can indicate, for example, the number of new residents in the facility at the given time. Generally, a higher number of new residents is correlated with a higher staffing need.

In some embodiments, the “pain” input 405 generally includes information relating to any pain issues reported or experienced by the residents. For example, this input may include the number of residents that complain of some pain, the type of the pain, the severity of the pain, and the like. As such pain reports can generally require additional attention, the “pain” input may be positively correlated with the staffing needs.

In one embodiment, the “treatments” input 405 can generally include information relating to specific treatments required or requested by the residents. For example, a resident may require treatments such as physical therapy, light exercise, or other activities. Generally, the residents may require assistance attending and/or engaging in these treatments. In some embodiments, the treatments input can generally indicate the number of residents needing such treatments, the number of such treatments that are needed in the window, the intensity or time required for such treatments, and the like. In an embodiment, the treatments input is positively correlated with the staffing needs.

In an embodiment, the “wounds” input 405 can include information relating to wounds that require treatment. In some embodiments, these wounds are included within the “treatment” category of input. In other embodiments, wounds are categorized separately due to the increased care they require. Wounds can generally include a wide variety of physical injuries, such as open sores, cuts, abrasions, and the like. In embodiments, the wounds input may include, for example, the number of such wounds that are being treated among the residents, the number of treatment events required for the wounds, the severity or time requirements of the treatments, and the like. Generally, higher values for the wounds input are correlated with a higher staffing need.

In some embodiments, the “acuity” input 405 can generally indicate the acuity of the residents, where “acuity” corresponds to care needs. In some embodiments, each resident has an acuity score generated by one or more systems (e.g., using machine learning), where higher acuity scores indicate increased care needs. In one embodiment, the acuity input can indicate, for example, the number of residents having an acuity score exceeding a defined threshold. Generally, higher values for the acuity input are correlated with higher staffing needs.

In the illustrated embodiment, the “census records” input 405 generally includes information relating to the overall population of residents, such as the total number of residents in the facility, the number of residents in each area or section, the room assignments, and the like.

As discussed above, in some embodiments, there may be other inputs used by the machine learning system 120. For example, in one such embodiment, the input 405 can include the aggregate (e.g., average) vitals of the residents. Vitals may generally include patient data such as the average weight, height, and the like. In some embodiments, the input can include diagnoses of the residents (e.g., the number with each diagnosis, the number with certain specified diagnoses, and the like). In at least one embodiment, in addition to age, the input includes more encompassing demographics of the residents.

In the illustrated workflow 400, the machine learning system 120 receives this input 405 (and possibly other data) to generate a set of uniform and aggregated resident characteristics 410. In some embodiments, as discussed above, the machine learning system 120 generates the resident characteristics 410 on a per-time and/or per-facility basis. For example, the machine learning system 120 may, for each window of a defined set of windows (e.g., for each shift), identify the relevant data in the input 405 (e.g., the data corresponding to the same window and facility), and generate resident characteristics 410 for this window and facility.

In some aspects, as discussed above, the machine learning system 120 can anonymize the input 405 in various ways. For example, rather than evaluating the specific conditions and needs of each individual resident, the machine learning system 120 may aggregate the data (e.g., on a facility-wide and/or shift-wide basis), as discussed above. This can allow the machine learning system 120 to learn the needs and complexities of a given shift at a given facility, without exposing the underlying resident data.

### Example Workflow for Determining Historical Staffing to Enable Generation of Optimized Resource Allocations

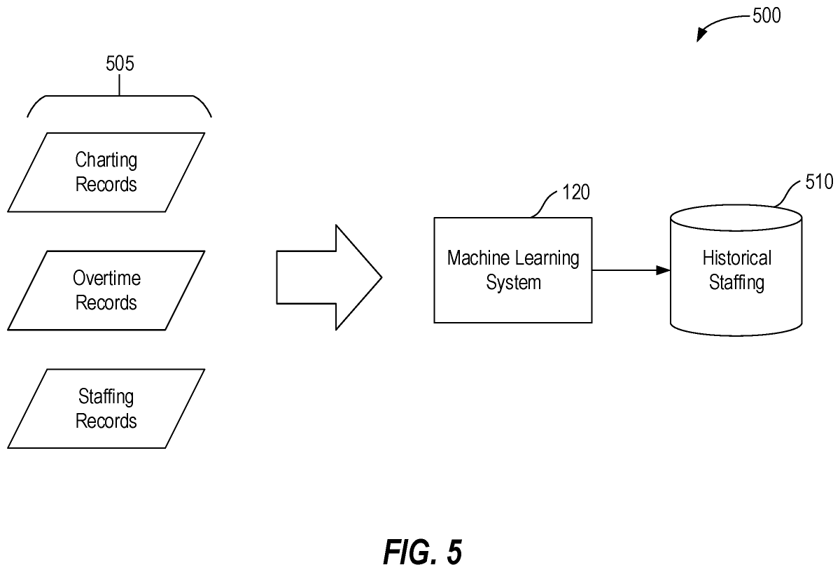

FIG. 5 depicts an example workflow 500 for determining historical staffing to enable generation of optimized resource allocations. In some embodiments, the workflow 500 is performed by a machine learning system, such as machine learning system 120 of FIG. 1.

In the illustrated workflow 500, a set of inputs 505 are received, evaluated, and/or aggregated by the machine learning system 120 in order to generate or determine overall historical staffing 510. In some embodiments, the inputs 505 are received from one or more facilities. For example, the inputs 505 may correspond to the staffing records 115 of FIG. 1.

In one embodiment, the historical staffing 510 correspond to the particular human resource allocation (e.g., staffing) used during a given shift and at a given facility. That is, the machine learning system 120 may parse the inputs 505 to extract the relevant data for a specific facility and window of time, and generate historical staffing 510 for that window and facility. The machine learning system 120 can similarly parse the inputs 505 to staffing for other periods or facilities. Though the illustrated example depicts the machine learning system 120 receiving relatively raw inputs 505 to determine the historical staffing 510, in some aspects, the inputs 505 are parsed by other systems (e.g., locally at each facility) to provide the historical staffing 510 to the machine learning system 120.

In some embodiments, the historical staffing 510 are used as target output for training the machine learning models discussed above. For example, during training, resident characteristics (e.g., resident characteristics 410 of FIG. 4) can be used as input to the model to generate some output allocation, and the corresponding historical staffing 510 can be used as target output against which the generated allocation is compared. Based on this comparison, a loss can be computed, and the loss can be used to refine the parameters of the model(s).

In the illustrated example, the inputs 505 include a wide variety of data. The depicted inputs 505 are included as examples for conceptual clarity, and the particular inputs used in various embodiments may differ depending on the particular application. That is, depending on the specific deployment and implementation of the techniques described herein, the machine learning system 120 may evaluate all, a subset of, or none of the depicted inputs 505. Additionally, in some aspects, other inputs may be used depending on the particular problem space and implementation.

In some embodiments, some or all of the inputs 505 may be stored or otherwise available in one or more repositories, such as within the systems of each facility. In the illustrated example, the inputs 505 include “Charting Records,” “Overtime Records,” and “Staffing Records,” each of which is discussed in more detail below, in turn.

In one embodiment, the “staffing records” input 505 can indicate, for one or more shifts, the individuals that were staffed for one or more facilities. For example, the staffing records may indicate the number of base caregivers (e.g., that perform tasks such as ADL assistance), the number of medication aides (e.g., those that can administer medications), and the like. In some embodiments, if this information is available, the machine learning system 120 can simply use it as the historical staffing 510. However, in some cases, the staffing records may be unavailable (e.g., because they do not exist). Similarly, in some cases, the machine learning system 120 may wish to modify the staffing records to improve the training process, as discussed above and in more detail below.

In an embodiment, the “charting records” input 505 may include information relating to which user(s) (e.g., which employees or contractors) performed which task(s) for one or more residents in the facility. For example, during or after a shift, caregivers may generally record the actions or tasks completed, such as assisting a given resident to eat, refreshing bandages on a resident's wound, and the like. In some embodiments, the machine learning system 120 can use the charting records input 505 to infer the staffing during each shift and at each facility. For example, the machine learning system 120 may retrieve the charting records corresponding to a given shift and facility, and evaluate them to identify the number of unique individuals that performed the tasks. Based on this data, the machine learning system 120 can generate historical staffing 510 for the shift to reflect the number of employees (and the number of each type of employee, in some aspects) that worked the shift.

In some embodiments, as discussed above, the machine learning system 120 can also evaluate the charting records input 505 to determine whether the historical staffing was appropriate. For example, the machine learning system 120 may determine the caregiver-to-resident ratio based on the charting records 505, and determine whether the number of caregivers was appropriate/adequate, inadequate, or excessive. In some embodiments, based on this determination, the machine learning system 120 can artificially adjust the historical staffing 510 for the shift, in order to train the machine learning model(s) to generate improved allocations. For example, if the machine learning system 120 determines that the caregiver-to-resident ratio is too high (e.g., with more caregivers than needed), the machine learning system 120 can reduce the number of caregivers used as target output for the shift when training the model.

In one embodiment, the “overtime records” input 505 can generally include information relating to overtime at the facilities. For example, as discussed above, this input can indicate the number of overtime hours worked collectively each week (e.g., as compared to the number of non-overtime hours worked), the percentage of employees that work overtime, the average overtime per employee, and the like. Based on this input 505, the machine learning system 120 may determine whether the staffing was appropriate, as discussed above. For example, if the overtime information exceeds one or more thresholds, the machine learning system 120 may determine that the staffing during the relevant shifts was inadequate, and therefore adjust the targeted historical staffing 510 upwards when training the model(s).

In the illustrated workflow 500, the machine learning system 120 uses this input 505 (and possibly other data) to generate accurate and useable historical staffing 510. In some embodiments, as discussed above, the machine learning system 120 generates the historical staffing 510 on a per-time and/or per-facility basis. For example, the machine learning system 120 may, for each window of a defined set of windows (e.g., for each shift), identify the relevant data in the input 505 (e.g., the data corresponding to the same window and facility), and generate historical staffing 510 for this window and facility.

The historical staffing 510 can then be used to train and/or refine machine learning model(s), as discussed above. In this way, the machine learning system 120 can train accurate and reliable models that generate optimized resource allocations given relevant input data, which significantly improves a wide variety of systems and functionality in the facilities.

### Example Method for Training Machine Learning Models to Generate Optimized Resource Allocations

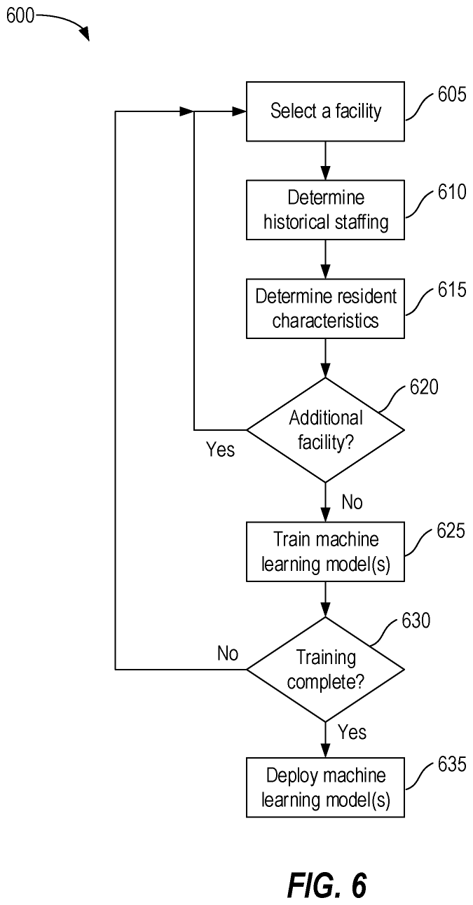

FIG. 6 is a flow diagram depicting an example method 600 for training machine learning models to generate optimized resource allocations. In some embodiments, the method 600 is performed by a machine learning system, such as machine learning system 120 of FIG. 1.

At block 605, the machine learning system selects a facility, from among the set of facilities that will be used to train the model(s). That is, if data from multiple different facilities will be used to train the models, the machine learning system can select one of these facilities for processing at block 605. In embodiments, the particular technique used to select the facility can vary depending on the particular implementation, as data from all such facilities will be processed in turn. Although the illustrated example depicts a sequential processing (e.g., where each facility is processed iteratively) for conceptual clarity, in embodiments, the machine learning system may process some or all of the facilities in parallel.

At block 610, the machine learning system determines the historical staffing of the selected facility. In at least one embodiment, the machine learning system determines this historical staffing by receiving and/or evaluating various staffing records, as discussed above, such as the overtime records, charting records, and the like. In at least one embodiment, the machine learning system determines the historical staffing at the selected facility using the workflow 500, discussed above with reference to FIG. 5.

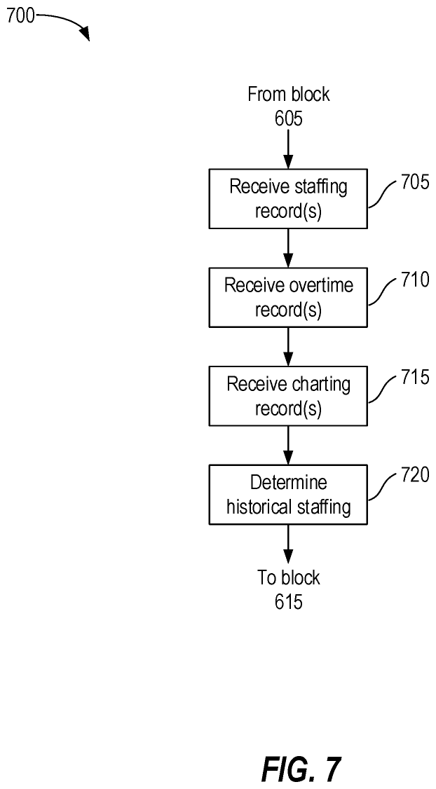

In some embodiments, determining the historical staffing includes determining the staffing at the selected facility during one or more discrete windows of time (e.g., work shifts). For example, the machine learning system may determine, for each respective shift in a given period, the number and skill mix of staff that worked that respective shift. In some embodiments, the machine learning system determines the staffing for a defined period, such as the last year, the period between the present and the last time training or refinement was performed, and the like. Various aspects of determining the historical staffing are described below with reference to FIG. 7.

At block 615, the machine learning system determines resident characteristics for the selected facility. In some embodiments, the resident characteristics are generally predictive of the amount of care needed in the facility. For example, the resident characteristics may indicate, for a given shift, the number and type of medication events, the number of ADL events, and the like. In at least one embodiment, the machine learning system determines the resident characteristics using the workflow 400, discussed above with reference to FIG. 4.

In some embodiments, determining the resident characteristics for a facility includes determining the characteristics at the selected facility during one or more discrete windows of time (e.g., work shifts). For example, the machine learning system may determine, for each respective shift in a given period, the specific resident characteristics and needs of that respective shift. In some embodiments, the machine learning system determines the characteristics for each shift during a defined period, such as the last year, the period between the present and the last time training or refinement was performed, and the like.

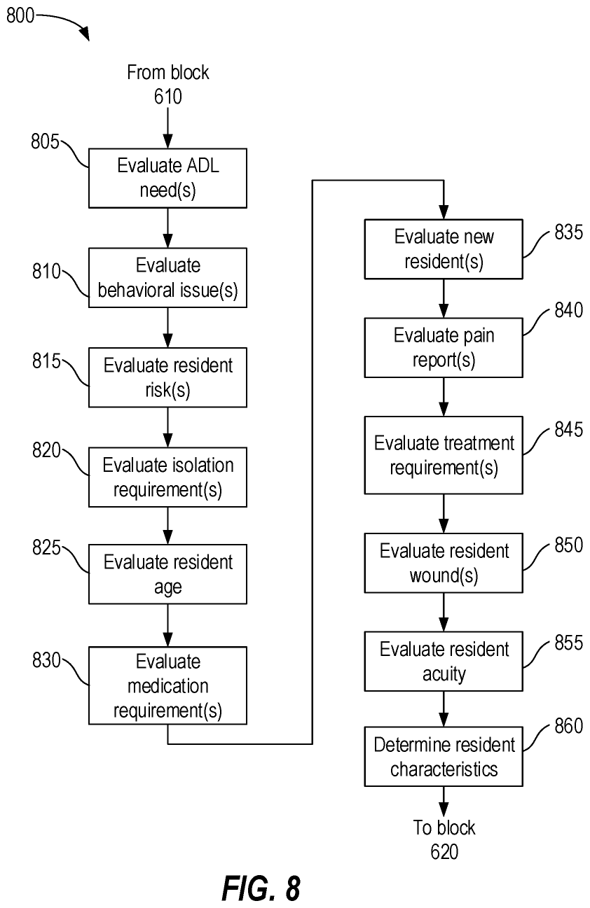

In at least one embodiment, the machine learning system determines the resident characteristics based on the same time windows used to define the historical staffing. For example, for each defined window of staffing (e.g., for each shift), the machine learning system can determine the corresponding resident characteristics during that window. Alternatively, for each set of resident characteristics, the machine learning system may determine the corresponding staffing for that time period. Various aspects of determining the resident characteristics are described below with reference to FIG. 8.

At block 620, the machine learning system determines whether there is at least one facility for which data has not yet been evaluated. If so, the method 600 returns to block 605. If all relevant facilities have been evaluated, the method 600 continues to block 625, where the machine learning system trains one or more machine learning models based on the determined historical staffing and the resident characteristics.

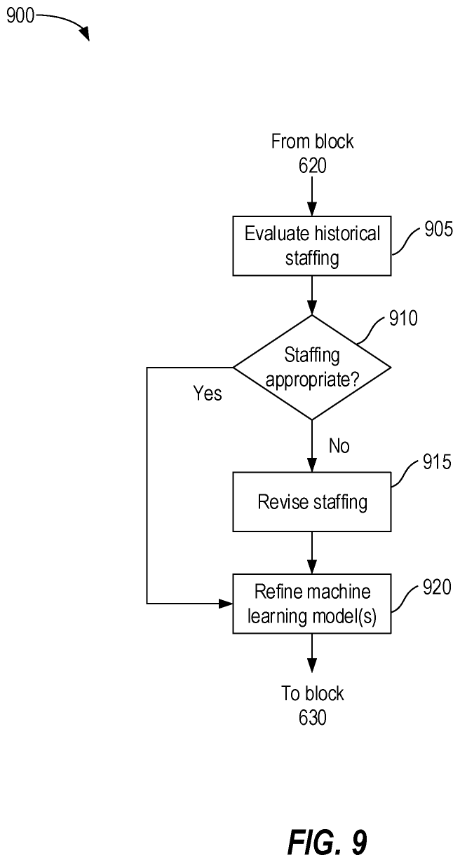

In at least one embodiment, to train the model(s), the machine learning system provides the resident characteristics from one or more shifts as input to the model(s) in order to generate an output resource allocation. This allocation can then be compared against the corresponding historical staffing for the shift in order to generate a loss, which is used to refine the model(s). This process can be repeated using data from any number of shifts and facilities in order to iteratively refine the model(s). In embodiments, the machine learning system may process the facility data separately or in batches. Various aspects of training the machine learning model(s) are described below with reference to FIG. 9.

At block 630, the machine learning system determines whether training is complete. In embodiments, this can include evaluation of a wide variety of termination criteria, including a maximum number of training iterations or epochs, a maximum time or computing resources spent training the model(s), determining whether there is any remaining training data, and the like. If training is not complete, the method 600 returns to block 605.

If the machine learning system determines that training is complete, the method 600 continues to block 635, where the machine learning system deploys the trained machine learning model(s) for inferencing. For example, in one embodiment, the machine learning system trains the model(s) at a central location using data from a variety of facilities. After training, the machine learning system can distribute the trained model to each individual facility, enabling optimized resource allocations to be generated locally within each facility, using the relevant local data.

### Example Method for Determining Historical Staffing to Enable Generation of Optimized Resource Allocations

FIG. 7 is a flow diagram depicting an example method 700 for determining historical staffing to enable generation of optimized resource allocations. In some embodiments, the method 700 is performed by a machine learning system, such as machine learning system 120 of FIG. 1. In one embodiment, the method 700 provides additional detail for block 610 of FIG. 6, where historical staffing is determined for training purposes.

In the illustrated example, the method 700 begins from block 605 of FIG. 6. At block 705, the machine learning system receives staffing records for a facility. For example, as discussed above, one or more facilities may store records indicating, for one or more prior shifts, the particular staffing of each shift (e.g., the number of caregivers, the number of nursing staff, the number of medication aides, and the like). In some embodiments, however, these records may be unavailable or not in existence.

At block 710, the machine learning system receives overtime records for the facility. As discussed above, these overtime records may generally include information related to overtime at the facility, such as the average number of overtime hours per employee, the number of overtime hours per week, and the like. As discussed above, excessive overtime hours may be indicative of understaffing in some aspects.

At block 715, the machine learning system receives charting records for the facility. In some embodiments, as discussed above, the charting records can generally indicate a variety of actions performed by caregivers, nurses, medication aides, and the like during a given shift. In some embodiments, the machine learning system can evaluate these charts to determine or infer the staffing during a given shift (e.g., if the actual staffing was not recorded). For example, the machine learning system can identify a set of employees, agents, or contractors that performed some charted action during the shift, and thereby determine the number of users (of each skill set) that were present and working during the shift.

At block 720, the machine learning system determines the historical staffing based on the received information, as discussed above. Generally, the historical staffing may indicate the staffing used during one or more shifts at one or more facilities. This staffing may indicate, for example, the total number of employees, the number of each type of employee (e.g., caregivers, medication specialists, etc.), and the like. The method 700 then continues to block 615 of FIG. 6.

### Example Method for Determining Resident Characteristics to Generate Optimized Resource Allocations

FIG. 8 is a flow diagram depicting an example method 800 for determining resident characteristics to generate optimized resource allocations. In some embodiments, the method 800 is performed by a machine learning system, such as machine learning system 120 of FIG. 1. In some embodiments, the method 800 provides additional detail for block 615 of FIG. 6.

In the illustrated example, the method 800 begins from block 610 of FIG. 6. At block 805, the machine learning system evaluates ADL need(s) of the residents in one or more facilities. As discussed above, the ADL needs may generally correspond to activities and/or daily living events at the facility that require assistance from one or more employees. For example, the ADL needs may indicate the number of residents needing assistance to eat, to transfer between seated positions, to walk, and the like. In some embodiments, the ADL needs are determined using data stored in one or more MDS systems, as discussed above.

At block 810, the machine learning system evaluates any reported behavioral issues at the facility or facilities. As discussed above, the behavioral issues can generally include a wide variety of concerns, including irritability, anger, fighting, yelling, and other actions that may require additional intervention from staff. In one aspect, at block 810, the machine learning system determines the number of reported issues, the number of residents with reported issues, and the like.

At block 815, the machine learning system evaluates a set of resident risk(s). As discussed above, the resident risks can generally include quantified risk of adverse events such as infection, falls, and the like. In one embodiment, these risks are represented by risk scores that can be generated using one or more machine learning models. The resident risks may be aggregated or anonymized, as discussed above. For example, the machine learning system may determine the number of residents with a risk score exceeding one or more defined threshold values, the average risk score of the facility, and the like.

At block 820, the machine learning system evaluates any isolation requirement(s) for the facility or facilities. As discussed above, isolation requirements may generally correspond to any quarantine or isolation required or used by the residents, such as due to immunocompromised status, infection, illness, and the like. In one embodiment, the machine learning system can evaluate this data to determine, for example, the total number of such residents in isolation.

At block 825, the machine learning system evaluates the age(s) of one or more residents in the facility. For example, the machine learning system can determine the average age of the residents. In some embodiments, other vitals can similarly be evaluated at this stage (e.g., the average weight or BMI, the number of residents having a weight or BMI above or below one or more thresholds, and the like).

At block 830, the machine learning system evaluates any medication requirements for the residents. For example, as discussed above, the machine learning system may determine the number of medication events (e.g., the number of times medication needs to be distributed to residents), the types of these medications (e.g., over-the-counter, psychotropic, opioids, and the like), and the like.

At block 835, the machine learning system evaluates the status of any new residents in the facility. For example, the machine learning system may determine the number of residents that have newly-arrived or moved into the facility (e.g., within the last 24 or 48 hours), or that will move in shortly before or during the upcoming shift for which the resource allocation is being generated.

At block 840, the machine learning system evaluates any pain reports from the residents. For example, the machine learning system may determine the number of such reports (overall or within defined windows), the severity of the reports, and the like.

At block 845, the machine learning system evaluates any treatment requirements for the residents. As discussed above, this may include determining the number of such events (overall or per shift), the type or complexity of these events, and the like.

At block 850, the machine learning system evaluates reported wound(s) needing care among the residents. For example, the machine learning system may determine the number of such wounds, the number of wound care events, the complexity of these events, and the like.

At block 855, the machine learning system evaluates resident acuities. For example, as discussed above, the machine learning system may identify the number of residents with an acuity score exceeding some threshold, the average acuity score, and the like.

At block 860, the machine learning system can determine the overall resident characteristics based on the above evaluations (in block 805 through 855). As discussed above, these resident characteristics can be informative of the staffing needs of a facility. Therefore, the machine learning system can use these data to train and refine the machine learning model(s) to generate optimized staffing allocations. The method 800 then returns to block 620 of FIG. 6.

### Example Method for Evaluating Historical Data to Improve Training Machine Learning Models

FIG. 9 is a flow diagram depicting an example method 900 for evaluating historical data to improve training machine learning models to generate optimized resource allocations. In some embodiments, the method 900 is performed by a machine learning system, such as machine learning system 120 of FIG. 1. In some embodiments, the method 900 provides additional detail for block 625 of FIG. 6.

In the illustrated example, the method 900 begins from block 620 of FIG. 6. At block 905, the machine learning system evaluates historical staffing at the facility. For example, the machine learning system may identify the historical staffing for a given shift and facility (e.g., determined at block 610 of FIG. 6) in order to determine whether the staffing was appropriate.

At block 910, the machine learning system evaluates the historical staffing in order to determine whether it was appropriate for the shift and facility. In some embodiments, as discussed above, the machine learning system can determine whether the staffing was appropriate based on information such as overtime hours for the facility, the charting ratio (e.g., the caregiver-to-resident ratio), and the like. If the machine learning system determines that the staffing was appropriate (e.g., not inadequate and not excessive), the method 900 proceeds to block 920, discussed below in more detail.

If, at block 910, the machine learning system determines that the staffing was not appropriate (e.g., because it was inadequate or excessive), the method 900 continues to block 915. At block 915, the machine learning system revises the historical staffing, for training purposes. For example, if the machine learning system determines that the staffing was inadequate in some way(s) (e.g., because there were fewer caregivers than needed or desired, or fewer medication specialists than warranted), the machine learning system can artificially increase the relevant staffing metrics to a more appropriate level. As discussed above, in some embodiments, the machine learning system may simultaneously determine that staffing was inadequate in some aspects (e.g., because additional caregivers were needed) and excessive in other aspects (e.g., because too many medication aides were staffed). In embodiments, the machine learning system can adjust the staffing data as needed to reflect more appropriate values.

At block 920, the machine learning system refines the machine learning model(s) based on the staffing. For example, as discussed above, the machine learning system may process a set of resident characteristics as input to the model in order to generate an output allocation. This output can then be compared against the corresponding historical staffing (or the modified staffing) from the same time window and facility, and the difference(s) can be used to refine the internal parameters of the model. In this way, the machine learning system iteratively learns to generate more accurate and optimized resource allocations. The method 900 then returns to block 630 of FIG. 6.

### Example Method for Generating Optimized Resource Allocations Using Machine Learning

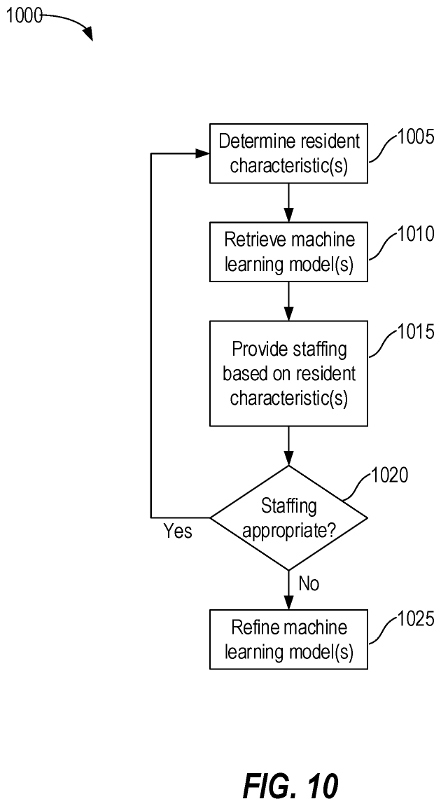

FIG. 10 is a flow diagram depicting an example method 1000 for generating optimized resource allocations using machine learning models. In some embodiments, the method 1000 is performed by a machine learning system, such as machine learning system 120 of FIG. 1.

At block 1005, the machine learning system determines a set of resident characteristics for some future window of time at a given facility. For example, for a shift that begins at some point in the future (e.g., tomorrow, next week, and the like), the machine learning system can determine or predict the various resident characteristics during that shift. In at least one embodiment, the machine learning system determines the resident characteristics using the workflow 400 of FIG. 4, and/or the method 800 of FIG. 8.

In at least one embodiment, the machine learning system determines the resident characteristics for the future window by extrapolating based on the resident characteristics at one or more current or prior windows. For example, the machine learning system may identify the resident characteristics during a comparable prior shift (e.g., covering the same time, such as from noon to midnight, during the same day of the week, and the like). In one embodiment, the machine learning system can simply use these prior characteristics as the resident characteristics for the future shift. In some embodiments, the machine learning system evaluates various data to predict some or all of the characteristics. For example, medication orders are typically documented and scheduled in advance, and information such as the resident's age and isolation status is generally known. In some embodiments, the machine learning system may use this data as the future resident characteristics.

At block 1010, the machine learning system retrieves one or more machine learning models to be used in processing the resident characteristics. For example, if the particular categories of data included in the resident characteristics may vary, the machine learning system may select a model that was trained for the relevant categories. In some embodiments, the machine learning system can select a facility-specific model. For example, the machine learning system may identify and retrieve a model that was trained and/or refined specifically for the facility corresponding to the resident characteristics (e.g., trained using data from the facility). In some embodiments, a general model is trained based on aggregated data from multiple facilities, and this model is specialized to each facility via fine-tuning using facility-specific data. In other embodiments, a single model is shared (unchanged) across all facilities.

At block 1015, the machine learning system provides staffing allocations based on the resident characteristics. For example, as discussed above, the machine learning system may process the resident characteristics using the model(s) to generate an optimized allocation, and use this allocation to assign staff members to the future shift. In embodiments, this may include automatically scheduling staff, requesting additional staff (e.g., from a contracting agency), suggesting a staffing schedule (e.g., to a manager, who can then approve or modify it), and the like.

At block 1020, at some future point after the relevant window has passed (e.g., after the shift has ended), the machine learning system determines whether the allocated staffing was appropriate. For example, the machine learning system may evaluate any written records (e.g., using natural language processing) to determine whether the staff or managers on the shift expressed any concerns related to the staffing. In some embodiments, one or more users (e.g., managers) can explicitly indicate whether the staffing was appropriate, and/or whether additional or fewer staff were needed. In at least one embodiment, to evaluate the staffing, the machine learning system can analyze data such as the charting records for the shift.

If the machine learning system determines that the staffing was appropriate, the method 1000 returns to block 1005 to generate a new staffing allocation for another future shift. If staffing was inappropriate, the method 1000 continues to block 1025, where the machine learning system optionally refines the selected machine learning model(s) based on this determination. For example, if the machine learning system determines that the generated allocation was insufficient, the machine learning system can refine the model(s) to suggest or allocate additional staff for the corresponding set of resident characteristics. In this way, the machine learning system can be automatically refined over time to ensure that the models continue to generate reliable and accurate resource allocations.

### Example Method for Implementing Optimized Resource Allocations Using Machine Learning

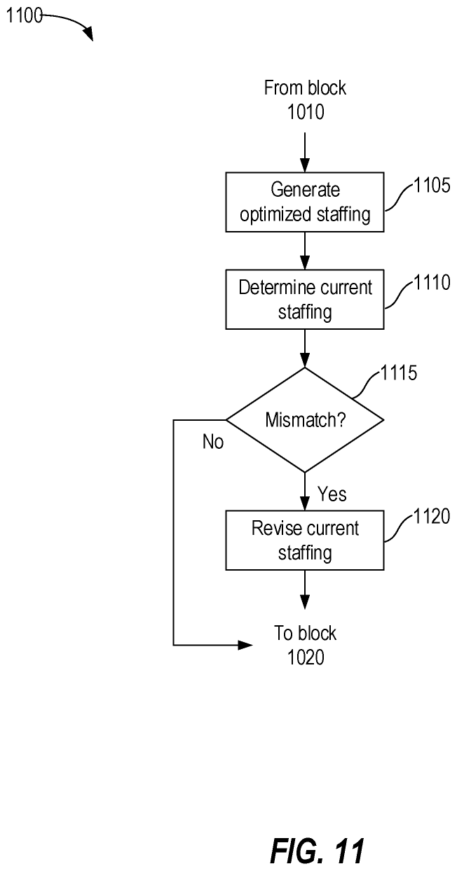

FIG. 11 is a flow diagram depicting an example method 1100 for implementing optimized resource allocations using machine learning models. In some embodiments, the method 1100 is performed by a machine learning system, such as machine learning system 120 of FIG. 1. In some embodiments, the method 1100 provides additional detail for block 1015 of FIG. 10.

The method 1100 begins at block 1105, where the machine learning system generates an optimized staffing allocation using one or more machine learning models, as discussed above. For example, the machine learning system may process a set of resident characteristics using model(s) trained to generate or suggest various resource allocations, such as staffing, based on resident characteristics for a future period of time (e.g., a future shift).

At block 1110, the machine learning system determines the currently-assigned staffing for the shift. For example, the machine learning system can determine the assigned number of base caregivers, the number of medication aides, and the like, that are scheduled to work the shift.

At block 1115, the machine learning system determines whether there is a mismatch between the currently-assigned staff and the optimized staffing allocation. If not, the method 1100 terminates and returns to block 1020 of FIG. 10. If the machine learning system determines that there is a mismatch, the method 1100 continues to block 1120, where the machine learning system revises the currently-assigned staffing. For example, the machine learning system may add additional staff for the shift, remove one or more staff members from the shift, and the like.

In some embodiments, as discussed above, the machine learning system may automatically revise the staffing (e.g., adding and/or removing staff automatically, without the need for manual confirmation). In some embodiments, some or all of the changes are provided to a user (e.g., a manager) for approval prior to being implemented. The method 1100 then returns to block 1020 of FIG. 10.

### Example Method for Generating Optimized Resource Mixes Using Machine Learning

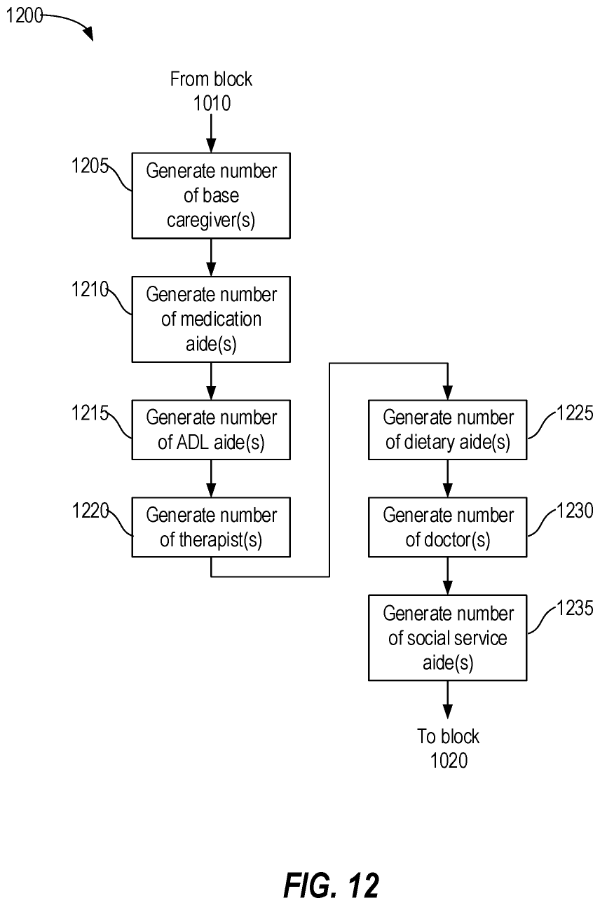

FIG. 12 is a flow diagram depicting an example method 1200 for generating optimized resource mixes using machine learning models. In some embodiments, the method 1200 is performed by a machine learning system, such as machine learning system 120 of FIG. 1. In one embodiment, the method 1200 provides additional detail for block 1015 of FIG. 10. In some embodiments, the method 1200 may additionally or alternatively provide additional detail for block 1105 of FIG. 11.

Generally, each block in the method 1200 may be optional, as the particular staffing or other resource allocations generated by the model may vary depending on the particular implementation. For example, some model(s) may be trained to optimize base caregiver staffing, while others optimize mediation aide staffing. Generally, each block in the method 1200 is performed by processing resident characteristics using one or more trained machine learning models.

At block 1205, the machine learning system generates an optimized number of base caregivers, as discussed above, using resident characteristics. As used herein, base caregivers may generally correspond to staff that are generally able to perform a variety of tasks and assistances without needing specialized training or authorization.

At block 1210, the machine learning system can generate an optimized number of mediation aides. As used herein, medication aides may generally be trained and/or authorized to administer various medications to residents.

Further, at block 1215, the machine learning system generates an optimized number of ADL aides. As used herein, ADL aides may generally include staff trained and/or authorized to perform certain ADL tasks, such as toileting and bathing, for the residents.

At block 1220, the machine learning system can generate an optimized number of therapists. Similarly, at block 1225, the machine learning system can generate an optimized number of dietary aides for the shift. As used herein, dietary aides can generally include any staff that assists with designing, preparing, and/or serving food to the residents.

At block 1230, the machine learning system generates an optimized number of doctors that should be present for the shift. Finally, at block 1235, the machine learning system generates an optimized number of social service aides that should be present during the shift. Generally, social service aides or assistants may work to connect the residents with relevant benefits and resources that may be available to them.

In some embodiments, as discussed above, the machine learning system may generate only a portion of these values, depending on the particular embodiment. For example, in some embodiments, the machine learning system can generate the number of base caregivers and the number of medication aides, without consideration of the other staff types (e.g., without generating a number of dietary aides.

### Example Method for Training Machine Learning Models to Generate Future Staffing

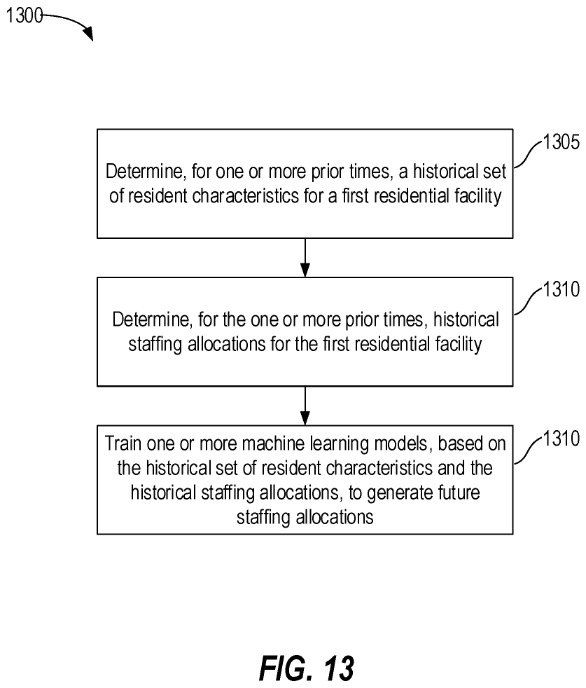

FIG. 13 is a flow diagram depicting an example method 1300 for training machine learning models to generate future staffing. In some embodiments, the method 1300 is performed by a machine learning system, such as machine learning system 120 of FIG. 1.

At block 1305, a historical set of resident characteristics for a first residential facility are determined for one or more prior times.

At block 1310, historical staffing allocations for the first residential facility are determined for the one or more prior times.

At block 1315, one or more machine learning models are trained, based on the historical set of resident characteristics and the historical staffing allocations, to generate future staffing allocations.

In some embodiments, the historical set of resident characteristics comprise at least one of: demographics of one or more residents in the first residential facility, diagnoses of one or more residents in the first residential facility, vitals of one or more residents in the first residential facility, medications used by one or more residents in the first residential facility, or physical assistance needed by one or more residents in the first residential facility.

In some embodiments, the historical staffing allocations indicate at least one of: a number of base caregivers for the first residential facility, or a number of medication aides of one or more residents in the first residential facility.

In some embodiments, the method 1300 further includes, prior to training the one or more machine learning models, determining whether the historical staffing allocations were appropriate for the first residential facility at the one or more prior times based on at least one of: a charting ratio at the first residential facility during the one or more prior times, or an amount of overtime at the first residential facility during the one or more prior times.

In some embodiments, the method 1300 further includes determining that the historical staffing allocations were appropriate, and training the one or more machine learning models by using the historical staffing allocations as target output for the historical set of resident characteristics.

In some embodiments, the method 1300 further includes determining that the historical staffing allocations were not appropriate, and training the one or more machine learning models by using increased staffing allocations, as compared to the historical staffing allocations, as target output for the historical set of resident characteristics.

In some embodiments, determining that the historical staffing allocations were not appropriate comprises at least one of: determining that the charting ratio exceeds a first defined threshold, wherein the charting ratio indicates a number of residents being cared for by each caregiver, or determining that the amount of overtime exceeds a second defined threshold.

In some embodiments, the method 1300 further includes deploying the one or more machine learning models to generate future staffing allocations for a second residential facility.

### Example Method for Using Trained Machine Learning Models to Modify Future Staffing

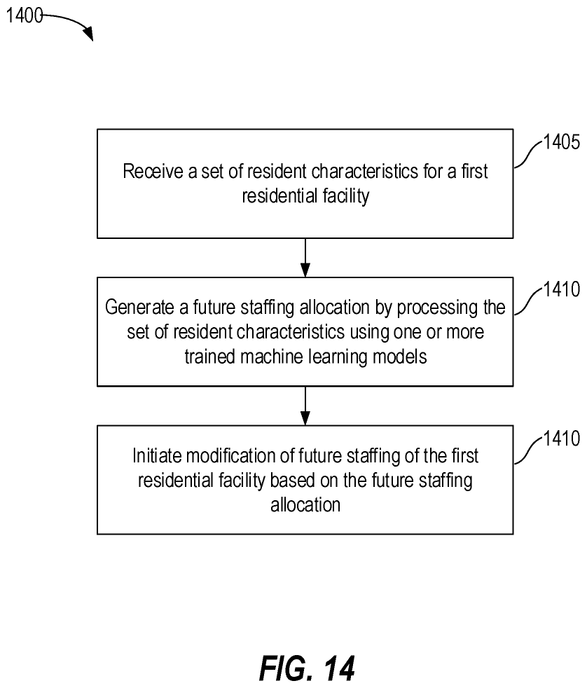

FIG. 14 is a flow diagram depicting an example method 1400 for using trained machine learning models to modify future staffing. In some embodiments, the method 1400 is performed by a machine learning system, such as machine learning system 120 of FIG. 1.

At block 1405, a set of resident characteristics is received for a first residential facility.

At block 1410, a future staffing allocation is generated by processing the set of resident characteristics using one or more trained machine learning models.

At block 1415, modification of future staffing of the first residential facility is initiated based on the future staffing allocation.

In some embodiments, the set of resident characteristics comprise at least one of: demographics of one or more residents in the first residential facility, diagnoses of one or more residents in the first residential facility, vitals of one or more residents in the first residential facility, medications used by one or more residents in the first residential facility, or physical assistance needed by one or more residents in the first residential facility.

In some embodiments, the future staffing allocation indicates at least one of: a number of base caregivers for the first residential facility, or a number of medication aides of one or more residents in the first residential facility.

In some embodiments, initiating modification of the future staffing comprises, upon determining that the future staffing of the first residential facility exceeds the future staffing allocation, reducing the future staffing.

In some embodiments, initiating modification of the future staffing comprises, upon determining that the future staffing allocation exceeds the future staffing of the first residential facility, increasing the future staffing.

In some embodiments, the future staffing allocation corresponds to a specific window of time, and the method 1400 further comprises: determining, after the specific window of time has passed, that staffing of the first residential facility was inadequate during the specific window of time, and refining the one or more trained machine learning models based on the determination that staffing was inadequate during the specific window of time.

### Example Processing System for Improved Machine Learning Models

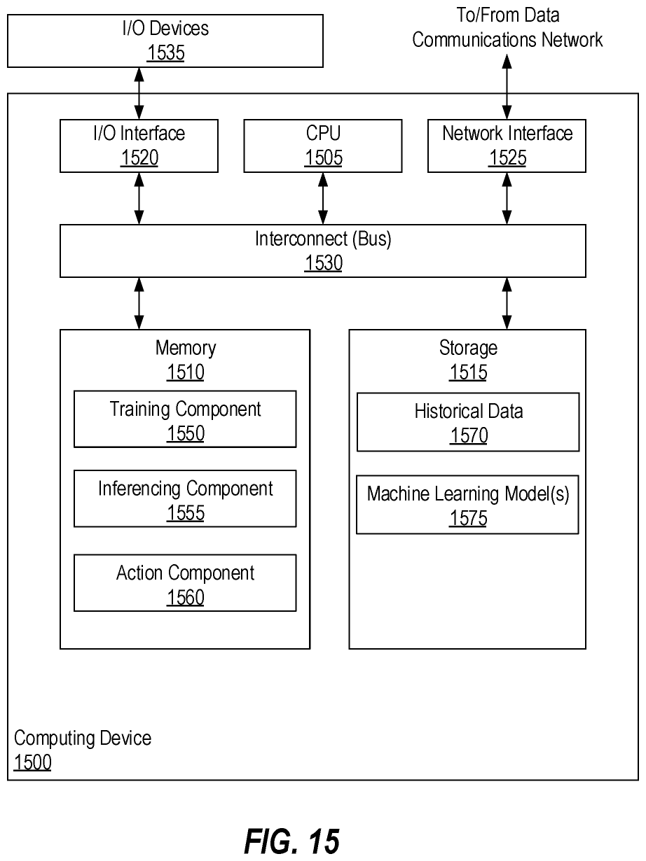

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In one embodiment, the computing device 1500 corresponds to the machine learning system 120 of FIG. 1.

As illustrated, the computing device 1500 includes a CPU 1505, memory 1510, storage 1515, a network interface 1525, and one or more I/O interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a training component 1550, an inferencing component 1555, an action component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software. In one embodiment, the training component 1550 is used to train the machine learning model(s), such as by using the method 600 of FIG. 6, and the inferencing component 1555 may be configured to use the models to generate optimized resource allocations using the trained models, such as by using the method 900 of FIG. 9. The action component 1560 may be configured to generate and/or initiate various actions based on the allocations, such as by increasing or decreasing the future staffing.

In the illustrated example, the storage 1515 includes historical data 1570 (which may correspond to resident characteristics and corresponding appropriate staffing, used train and/or evaluate the models), as well as one or more machine learning model(s) 1575. Although depicted as residing in storage 1515, the historical data 1570 and machine learning model(s) 1575 may be stored in any suitable location, including memory 1510.

### Example Clauses

Implementation examples are described in the following numbered clauses:

Clause 1: A method, comprising: for one or more prior times, determining a historical set of resident characteristics for a first residential facility; determining historical staffing allocations for the first residential facility; and training one or more machine learning models, based on the historical set of resident characteristics and the historical staffing allocations, to generate future staffing allocations, comprising: generating an output by processing the historical set of resident characteristics using the one or more machine learning models; and computing a loss based on the output and the historical staffing allocations.

Clause 2: The method of Clause 1, wherein the historical set of resident characteristics comprise at least one of: (i) demographics of one or more residents in the first residential facility; (ii) diagnoses of one or more residents in the first residential facility; (iii) vitals of one or more residents in the first residential facility; (iv) medications used by one or more residents in the first residential facility; or (v) physical assistance needed by one or more residents in the first residential facility.

Clause 3: The method of any one of Clauses 1-2, wherein the historical staffing allocations indicate at least one of: (i) a number of base caregivers for the first residential facility; or (ii) a number of medication aides of one or more residents in the first residential facility.

Clause 4: The method of any one of Clauses 1-3, further comprising, prior to training the one or more machine learning models, determining whether the historical staffing allocations were appropriate for the first residential facility at the one or more prior times based on at least one of: (i) a charting ratio at the first residential facility during the one or more prior times; or (ii) an amount of overtime at the first residential facility during the one or more prior times.

Clause 5: The method of any one of Clauses 1-4, further comprising: determining that the historical staffing allocations were appropriate; and training the one or more machine learning models by using the historical staffing allocations as target output for the historical set of resident characteristics.

Clause 6: The method of any one of Clauses 1-5, further comprising: determining that the historical staffing allocations were not appropriate; and training the one or more machine learning models by using increased staffing allocations, as compared to the historical staffing allocations, as target output for the historical set of resident characteristics.

Clause 7: The method of any one of Clauses 1-6, wherein determining that the historical staffing allocations were not appropriate comprises at least one of: determining that the charting ratio exceeds a first defined threshold, wherein the charting ratio indicates a number of residents being cared for by each caregiver; or determining that the amount of overtime exceeds a second defined threshold.

Clause 8: The method of any one of Clauses 1-7, further comprising: deploying the one or more machine learning models to generate future staffing allocations for a second residential facility.

Clause 9: A method, comprising: receiving a set of resident characteristics for a first residential facility; generating a future staffing allocation by processing the set of resident characteristics using one or more trained machine learning models; and initiating modification of future staffing of the first residential facility based on the future staffing allocation.

Clause 10: The method of Clause 9, wherein the set of resident characteristics comprise at least one of: (i) demographics of one or more residents in the first residential facility; (ii) diagnoses of one or more residents in the first residential facility; (iii) vitals of one or more residents in the first residential facility; (iv) medications used by one or more residents in the first residential facility; or (v) physical assistance needed by one or more residents in the first residential facility.

Clause 11: The method of any one of Clauses 9-10, wherein the future staffing allocation indicates at least one of: (i) a number of base caregivers for the first residential facility; or (ii) a number of medication aides of one or more residents in the first residential facility.

Clause 12: The method of any one of Clauses 9-11, wherein initiating modification of the future staffing comprises, upon determining that the future staffing of the first residential facility exceeds the future staffing allocation, reducing the future staffing.

Clause 13: The method of any one of Clauses 9-12, wherein initiating modification of the future staffing comprises, upon determining that the future staffing allocation exceeds the future staffing of the first residential facility, increasing the future staffing.

Clause 14: The method of any one of Clauses 9-13, wherein the future staffing allocation corresponds to a specific window of time, the method further comprising: determining, after the specific window of time has passed, that staffing of the first residential facility was inadequate during the specific window of time; and refining the one or more trained machine learning models based on the determination that staffing was inadequate during the specific window of time.

Clause 15: A system, comprising: a memory comprising computer-executable instructions; and one or more processors configured to execute the computer-executable instructions and cause the processing system to perform a method in accordance with any one of Clauses 1-14.

Clause 16: A system, comprising means for performing a method in accordance with any one of Clauses 1-14.

Clause 17: A non-transitory computer-readable medium comprising computer-executable instructions that, when executed by one or more processors of a processing system, cause the processing system to perform a method in accordance with any one of Clauses 1-14.

Clause 18: A computer program product embodied on a computer-readable storage medium comprising code for performing a method in accordance with any one of Clauses 1-14.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 120) or related data available in the cloud. For example, the machine learning system 120 could execute on a computing system in the cloud and train and/or use machine learning models. In such a case, the machine learning system 120 could train models to generate improved resource allocations, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of training machine learning models, comprising:
for one or more prior times,
determining a historical set of resident characteristics for a first residential facility;
determining historical staffing allocations for the first residential facility; and
training one or more machine learning models, based on the historical set of resident characteristics and the historical staffing allocations, to generate future staffing allocations, comprising:
generating an output by processing the historical set of resident characteristics using the one or more machine learning models; and
computing a loss based on the output and the historical staffing allocations.

2. The method of claim 1, wherein the historical set of resident characteristics comprise at least one of:
(i) demographics of one or more residents in the first residential facility;
(ii) diagnoses of one or more residents in the first residential facility;
(iii) vitals of one or more residents in the first residential facility;
(iv) medications used by one or more residents in the first residential facility; or
(v) physical assistance needed by one or more residents in the first residential facility.

3. The method of claim 1, wherein the historical staffing allocations indicate at least one of:
(i) a number of base caregivers for the first residential facility; or
(ii) a number of medication aides of one or more residents in the first residential facility.

4. The method of claim 1, further comprising, prior to training the one or more machine learning models, determining whether the historical staffing allocations were appropriate for the first residential facility at the one or more prior times based on at least one of:
(i) a charting ratio at the first residential facility during the one or more prior times; or
(ii) an amount of overtime at the first residential facility during the one or more prior times.

5. The method of claim 4, further comprising:
determining that the historical staffing allocations were appropriate; and
training the one or more machine learning models by using the historical staffing allocations as target output for the historical set of resident characteristics.

6. The method of claim 4, further comprising:
determining that the historical staffing allocations were not appropriate; and
training the one or more machine learning models by using increased staffing allocations, as compared to the historical staffing allocations, as target output for the historical set of resident characteristics.

7. The method of claim 6, wherein determining that the historical staffing allocations were not appropriate comprises at least one of:
determining that the charting ratio exceeds a first defined threshold, wherein the charting ratio indicates a number of residents being cared for by each caregiver; or
determining that the amount of overtime exceeds a second defined threshold.

8. The method of claim 1, further comprising:
deploying the one or more machine learning models to generate future staffing allocations for a second residential facility.

9. A method of configuring facilities using machine learning, comprising:
receiving a set of resident characteristics for a first residential facility;
generating a future staffing allocation by processing the set of resident characteristics using one or more trained machine learning models; and
initiating modification of future staffing of the first residential facility based on the future staffing allocation.

10. The method of claim 9, wherein the set of resident characteristics comprise at least one of:
(i) demographics of one or more residents in the first residential facility;
(ii) diagnoses of one or more residents in the first residential facility;
(iii) vitals of one or more residents in the first residential facility;
(iv) medications used by one or more residents in the first residential facility; or
(v) physical assistance needed by one or more residents in the first residential facility.

11. The method of claim 9, wherein the future staffing allocation indicates at least one of:
(i) a number of base caregivers for the first residential facility; or
(ii) a number of medication aides of one or more residents in the first residential facility.

12. The method of claim 9, wherein initiating modification of the future staffing comprises, upon determining that the future staffing of the first residential facility exceeds the future staffing allocation, reducing the future staffing.

13. The method of claim 9, wherein initiating modification of the future staffing comprises, upon determining that the future staffing allocation exceeds the future staffing of the first residential facility, increasing the future staffing.

14. The method of claim 9, wherein the future staffing allocation corresponds to a specific window of time, the method further comprising:
determining, after the specific window of time has passed, that staffing of the first residential facility was inadequate during the specific window of time; and
refining the one or more trained machine learning models based on the determination that staffing was inadequate during the specific window of time.

15. A non-transitory computer-readable storage medium comprising computer-readable program code that, when executed using one or more computer processors, performs an operation comprising:
receiving a set of resident characteristics for a first residential facility;
generating a future staffing allocation by processing the set of resident characteristics using one or more trained machine learning models; and
initiating modification of future staffing of the first residential facility based on the future staffing allocation.

16. The non-transitory computer-readable storage medium of claim 15, wherein the set of resident characteristics comprise at least one of:
(i) demographics of one or more residents in the first residential facility;
(ii) diagnoses of one or more residents in the first residential facility;
(iii) vitals of one or more residents in the first residential facility;
(iv) medications used by one or more residents in the first residential facility; or
(v) physical assistance needed by one or more residents in the first residential facility.

17. The non-transitory computer-readable storage medium of claim 15, wherein the future staffing allocation indicates at least one of:
(i) a number of base caregivers for the first residential facility; or
(ii) a number of medication aides of one or more residents in the first residential facility.

18. The non-transitory computer-readable storage medium of claim 15, wherein initiating modification of the future staffing comprises, upon determining that the future staffing of the first residential facility exceeds the future staffing allocation, reducing the future staffing.

19. The non-transitory computer-readable storage medium of claim 15, wherein initiating modification of the future staffing comprises, upon determining that the future staffing allocation exceeds the future staffing of the first residential facility, increasing the future staffing.

20. The non-transitory computer-readable storage medium of claim 15, wherein the future staffing allocation corresponds to a specific window of time, the operation further comprising:
determining, after the specific window of time has passed, that staffing of the first residential facility was inadequate during the specific window of time; and
refining the one or more trained machine learning models based on the determination that staffing was inadequate during the specific window of time.
